(19) United States
(12) Patent Application Publication
(10) Pub. No.: US 2025/0037845 A1
(43) Pub. Date: Jan. 30, 2025

(54) HEALTHCARE REFERRAL EVENT NOTIFICATIONS

(71) Applicant: Electronic Referral Manager Inc., Mt. Pleasant, SC (US)

(72) Inventors: Richard Hammer, Tucson, AZ (US); Ivan Nolasco, Santo Domingo (DO); David Bongiovanni, Mt. Pleasant, SC (US)

(73) Assignee: Electronic Referral Manager Inc., Mt. Pleasant, SC (US)

(21) Appl. No.: 18/227,395

(22) Filed: Jul. 28, 2023

(51) Int. Cl. G16H 40/20; G16H 10/60

(52) U.S. Cl. CPC G16H 40/20; G16H 10/60

(57) ABSTRACT

Embodiments include a method for sending a notification to a recipient, the method comprising determining a status of a referral event responsive to determining that the status of the referral event, determining communication preferences of the recipient, populating dynamic variables of a message template with corresponding data associated with the referral event to generate a communication, sending the communication including information associated with the referral event based on the communication preference of the recipient, logging a communication error responsive to a communication error, and logging that the communication is sent successfully responsive to successfully sending the communication.

### BACKGROUND

The present invention relates to managing healthcare referrals, and more specifically, to providing patients, healthcare providers and their staff members with information about referrals.

In the healthcare industry, patients are often recommended or referred to a healthcare provider by another healthcare provider. These referrals are often made by, for example, a primary care provider to a specialist provider—a dermatologist in this example. For example, if a patient experiences a skin condition, the primary care provider may refer the patient to a dermatologist for more specialized care. The referral results in an appointment for the patient with a specialist.

### SUMMARY

Embodiments of the present invention are directed to a method for sending a notification to a recipient, the method comprising determining a status of a referral event responsive to determining that the status of the referral event, determining communication preferences of the recipient, populating dynamic variables of a message template with corresponding data associated with the referral event to generate a communication, sending the communication including information associated with the referral event based on the communication preference of the recipient, logging a communication error responsive to a communication error, and logging that the communication is sent successfully responsive to successfully sending the communication.

Embodiments of the present invention for sending a referral event status update, the method comprise determining whether a user has a system account associated with the user, determining whether a facsimile (fax) number associated with the user is saved in the system, determining whether fax reporting is enabled in the user preferences, determining whether referral event status reports are enabled in the user preferences, responsive to determining that fax reporting and referral status reports are enabled in the user preferences, generating a summary referral report, retrieving the consultation report from system account or electronic health records (EHR) system, entering the summary referral report into a fax queue, and sending the summary referral report via fax to the user and the referring provider.

Embodiments of the present invention comprise a method for sending a report by a system, the method comprises adding the report to a referral by polling an electronic health records (EHR) system to determine whether the report is available, retrieving referral information, wherein the referral information includes information associated with a referring provider, determining communication preferences of the recipient, and sending the communication including information associated with the referral with respect to the communication preference.

### BRIEF DESCRIPTION OF THE DRAWINGS

FIG. 1 illustrates a block diagram of an example computer system.

FIG. 2 illustrates an exemplary method for sending referral status updates.

FIG. 3 illustrates an exemplary method for sending a status report via fax.

FIG. 4 illustrates an exemplary method for sending a consultation report.

FIG. 5 illustrates an exemplary method for sending consultation reports internally in the system.

FIG. 6 illustrates an exemplary method for sending a consultation report via fax.

FIG. 7 illustrates an exemplary method for sending automated periodic reports via fax.

FIG. 8 illustrates an exemplary method of sending status updates.

FIG. 9 illustrates an exemplary embodiment of a method for sending reminders and follow up messages to a patient.

FIG. 10 illustrates an exemplary embodiment of a method for sending appointment instructions.

FIG. 11 includes an exemplary method for sending a patient communication.

FIG. 12 illustrates an exemplary embodiment of a method for receiving patient responses via SMS.

FIG. 13 illustrates an exemplary method for receiving patient responses via email.

### DETAILED DESCRIPTION

Although this disclosure includes a detailed description of a computing environment, the teachings herein are not limited to the described computing environment. In embodiments, any implementation of a computer environment may be used whether now understood or later developed.

The referral system described herein provides a system that automatically notifies patients and healthcare providers about the status of referral events. A referral event may include a referral and an appointment with a healthcare provider. Notifications may include reports.

For example, if a patient has a referral appointment, the patient may receive a message notifying the patient of the time and place of the referral appointment. The referral methods of the system generate referral status updates based on the referral information stored in the database. The referral portion of the system uses notification rules and user preferences to determining the timing and frequency of updates based on a referral status. The rules or trigger events may include, for example, when a referral is received by the system, when a referral is sent by the system, and when a referral status has changed. A referral status may include, for example, draft, new, in-review, accepted, declined, scheduled, attended, no show, completed, canceled, archived sub-statutes. The sub-status trigger includes when consult reports are attached to referrals and status moved to completed. Other trigger events include, for example, a referral message is received, a referral alert is triggered, appointment scheduled, appointment status changed (ex. confirmed, canceled, rescheduled, no show, completed).

Notifications are also sent to the healthcare providers associated with the referral. The system generates referral status updates based on the referral information stored in the database or memory. The database includes patient and provider information such as, for example, for patients, patient information including name of the patient contact information, appointment schedules, and communication preferences. The database is integrated with electronic health records such that appointment schedules are up-to-date and accurate. The database also allows healthcare providers to manage patient information, update appointment schedules, and customize message templates.

The updates are sent to healthcare providers via various communication channels such as, for example, short message system (SMS), phone, fax, email, or via an internal system notification feature available to users having accounts or access to the system. The system offers improved communication between healthcare providers, reduces patient wait times, and increases the efficiency of the referral process.

The system provides automated status updates to referring providers who have accounts or access to the system and to providers who do not have access to the system based on rules defined by the user as referrals advance through a workflow. The automated notifications are sent electronically if the referring providers have a system account or access to the system. Alternatively, the notifications may be sent via facsimile (fax).

In embodiments, if electronic health records (EHR) system integrations are available, the system may automatically retrieves consultation reports from the EHR of the receiving provider, update the status of a referral to completed, update the referral order to closed in the referring provider EHR, send the consultation report from the receiving provider to the referring provider EHR, or queue the consultation report for the referring provider to review in the EHR. The consultation report (consult report) is a progress note or report such as, for example, a radiology report or other document written by a specialist healthcare provider after the examination, evaluation or diagnostic testing of the patent. The consultation report is sent to the referring provider outlining the specialist findings of the specialist, diagnoses and, a recommended treatment plan for the patient.

If there are EHR integrations available, the system 100 may automatically (without user action) retrieve consultation reports from the EHR of a receiving provider, update the status as completed in the system 100, update the referral order to closed in the EHR of the referring provider. In embodiments, the system may send a consultation report electronically to the EHR of the referring provider, and queue the consultation report for the provider to review in an EHR.

In a referral system, information about healthcare providers is input into the system from a variety of sources including, for example, external databases and proprietary databases. These sources are combined into a primary database that may include data from, for example, the Centers for Medicare and Medicaid Services (CMS) referral data, and CMS National Provider Identifier (NPI) data. The use of such databases may be used to match a patient with a provider.

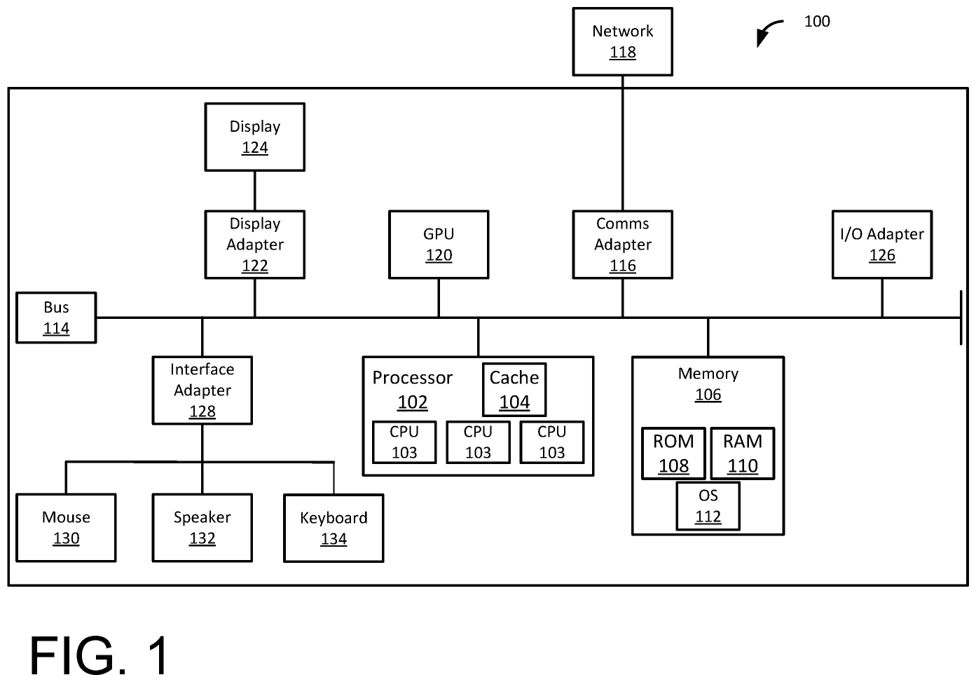

FIG. 1 illustrates a block diagram of an example computer system 100. In embodiments, a microprocessor is arranged in a personal computer, workstation, minicomputer, or mainframe computer. Embodiments system 100 a mobile device or part of a mobile device.

The system (computer) 100 includes processor 102 communicatively connected to an input/output (I/O) adapter 126, memory 106, a communications adapter 116, an interface adapter 128, a display adapter 122, and a graphics processing unit (GPU) 120 via a system bus 114.

The processor 102 includes any number of central processing units (CPU) 103 and a cache memory 104. The CPU 102 is operable to perform any number of processing tasks by executing program instructions. In embodiments the processor 102 may include any number of suitable auxiliary processors or microprocessors. The processor 102 may include a cache memory 104 that is often operative to increase the speed of processing tasks performed by the processor 102.

The I/O adapter 126 connects a variety of input and output devices to the computer 100. The memory 106 may include any type of suitable memory such as, for example, magnetic, solid state, disk-based storage, drum-based storage, random access memory (RAM) 110, and read only memory (ROM) 108. The operating system of the system 100 may be stored in the memory 106. The communications adapter 116 is operative to facilitate communications between the system 100 and a network 118. The network 118 may include, for example, a local area network, a wide area network or the Internet. The system 100 may connect to the network 118 via, for example, a wired or wireless connection. The display adapter 122 is operative to communicably connect a display 124 to the system 100. The display may include any type of suitable display such as, for example, a desktop display, a touch screen, or a mobile display. The GPU 120 includes a processor that is operative to generate graphical data that may be sent to the display 124 for presentation to a user. The interface adapter 128 provides an interface between peripheral devices and the system bus 114. The peripheral devices may include, for example, a mouse 130, a speaker 132, and a keyboard 134, sensors, and actuators, among others.

The memory 106 may store computer-readable and computer-executable instructions. The instructions implement logical functions. The operating system 112 may control the operations of the system 100 and the functions of other software programs or operations.

Memory can also include program instructions for engine 1, configured to improve the wellness of a user.

According to some embodiments, computer 100 can include a mobile communications adapter 123. Mobile communications adapter 123 can include GPS, cellular, mobile, and/or other communications protocols for wireless communication.

In some embodiments, computer 100 can further include communications adapter 116 for coupling to a network 118.

The healthcare referral process often involves a first healthcare provider providing a referral to a second healthcare provider. The second healthcare provider provides additional services to the patient that may not be available from the first healthcare provider.

It is challenging to provide information about a referral to the parties associated with the referral. Indeed, in some examples, the patient may be notified when a referral appointment is scheduled, the notification may include, for example, a name of the specialist provider, a time of the appointment and an option for the patient to reply to the notification to, for example, cancel or reschedule an appointment, when a referral is sent by referring providers. With respect to the communications, a notification may be sent to the specialist provider including information about the primary or referring practitioner and the patient. A notification may be sent to the referring or primary practitioner that may include, for example, information about the referral appointment, and a status that may include whether the patient has meet with the specialist provider.

The embodiments described herein provide a system 100 that includes a plurality of user accounts. Providers and users may have accounts in the system 100. When sending notifications to patients and providers who have access to the system, the notifications may be sent by the system to any one of the various accounts such that the user may be notified of the referral by messages sent internally in the system. In this regard, the user may log into the system to retrieve the notification. In other embodiments, the user may be sent a message via email or text that provides the information or provides a link that allows the user to access the notification information.

One challenge for sending notifications to providers is that some providers may not have an account in the system or have access to the system. The notifications may be sent to some of these providers via email or other options such as, for example, the use of a facsimile machine or system account. The use of a facsimile system to provide notifications to practitioners may provide some challenges. Examples of challenges include faxes not being transmitted successfully due to phone line problems, busy signals, faxes not delivered to the appropriate recipient, and printed faxes not delivered to a recipient in a timely matter.

The system 100 described herein offers a variety of approaches to providing referral notifications and information to healthcare practitioners and patients.

The system 100 includes referral data that includes NPI provider data (healthcare provider data) and NPI organization data (organizations such as, for example, associated medical practices, hospitals, etc.). The referral data may include any number of types of data. For example, the referral data may include data in a healthcare provider (provider) database that may be retrieved from the NPI data. The healthcare provider database includes the name of a provider, the specialty of a provider, a hospital associated with the provider, who the provider sends referrals to, and who the provider receives referrals from. Other data includes whether the provider will accept new patients, the insurance networks accepted by the provider, the specialties of the provider, common procedures performed by the provider, demographic information, average wait and response times, the language spoken by the provider, the gender of the healthcare provider, International Classification of Diseases, 10^{th} revision (ICD-10), and the geographic location of the provider. The IDC-10 is a classification and coding system for medical diagnoses and procedures managed by the World Health Organization. The healthcare provider database may also include, for example, average wait and response times, healthcare provider ratings and reviews, fees for services, associations with groups, education, specialties, titles, and credentials. Other categories that may be included include Current Procedural Terminology (CPT) codes which is a classification and coding system for medical procedures and services managed by the American Medical Association. Taxonomy codes may be included; taxonomy codes are a classification and coding system for healthcare provider specialties managed by the National Uniform Claim Committee (NUCC). Other codes may include, for example, Systemized Nomenclature of Medicine Clinical Terms classification and coding system for medical terms. This may include conditions and procedures and is managed by the International Health Terminology Standards Development Organization (IHTSDO).

The present invention may include a computer program, a method, system, and/or a computer program product in any level of integration. A computer program product can comprise of any type of computer readable storage medium that includes computer readable program instructions that causes a processor to implement some or all aspects of the invention.

The computer readable storage medium may include any suitable device that may store instructions for use by a computer, processor or other similar device. The computer readable storage medium may include, for example a random access memory, a read only memory, a flash memory, a magnetic or light readable disk. A computer readable storage medium as used herein does not include transitory signals such as, for example, electromagnetic waves.

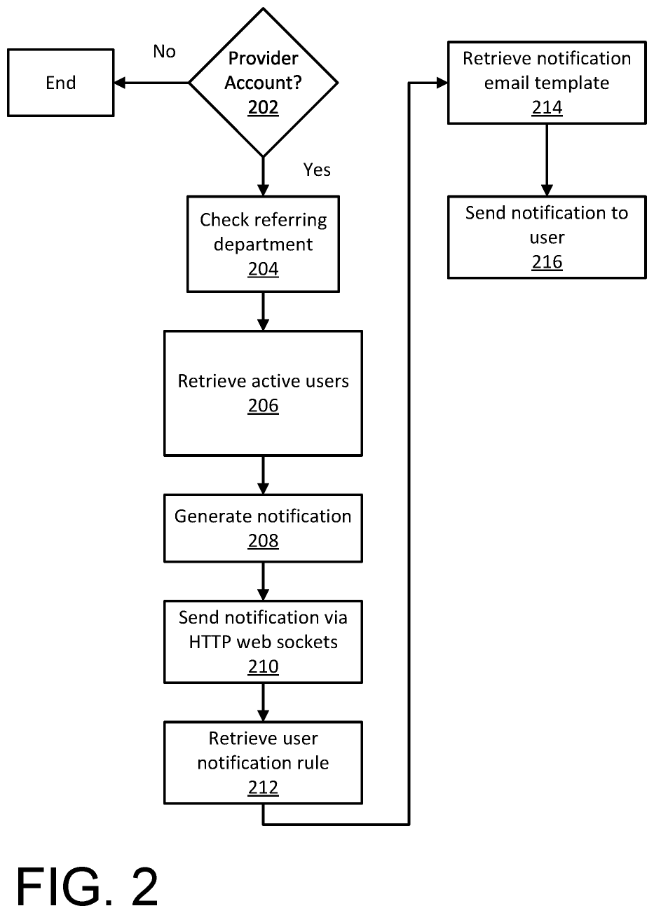

FIGS. 2-8 provide exemplary methods for managing referrals and referral notifications for healthcare providers.

Notifications send referral status updates to healthcare providers via various communications channels, such as, for example SMS, email, fax, or notifications provided to users of the system 100 when a user has access or an account on the system 100. The notifications may include user specific preferences to select the preferred communications for the user.

The system 100 updates referral status or appointment dates in the system 100. The status update may be sent to the referring provider via the system when the user has a system account or access to the system. In this regard, the user may receive a notification via text or email that the user should access the system to view or receive the updated referral status. In embodiments, the updated referral status may be sent to the user via fax. If system integrations are enabled for the user account, the status update may be sent to external EHRs.

The database or memory of the system 100 includes referral information, internal provider account information, and external provider contacts.

FIG. 2 illustrates an exemplary method for sending referral status updates internally in the system 100 to users who have access to the system 100. In this regard, the system 100 is operative to determine whether a user has a provider account or access to the system 100 in block 202. If yes, the system 100 identifies or checks the referring department found in the referral in block 204. In block 206 the system 100 retrieves the active users in the referring account assigned to the referring department. In block 208 the system 100 generates a notification. In block 210 a notification is sent via hypertext transfer protocol (HTTP) web sockets to the user so that the browser page of the user refreshes the referral status and the referral counts per status automatically.

In block 212, the system 100 retrieves a user notification rule that corresponds to the referral status. In block 214, the system 100 retrieves a notification email template that corresponds to a notification rule associated with the provider account. In block 216, a notification may be sent to the user via, for example, email.

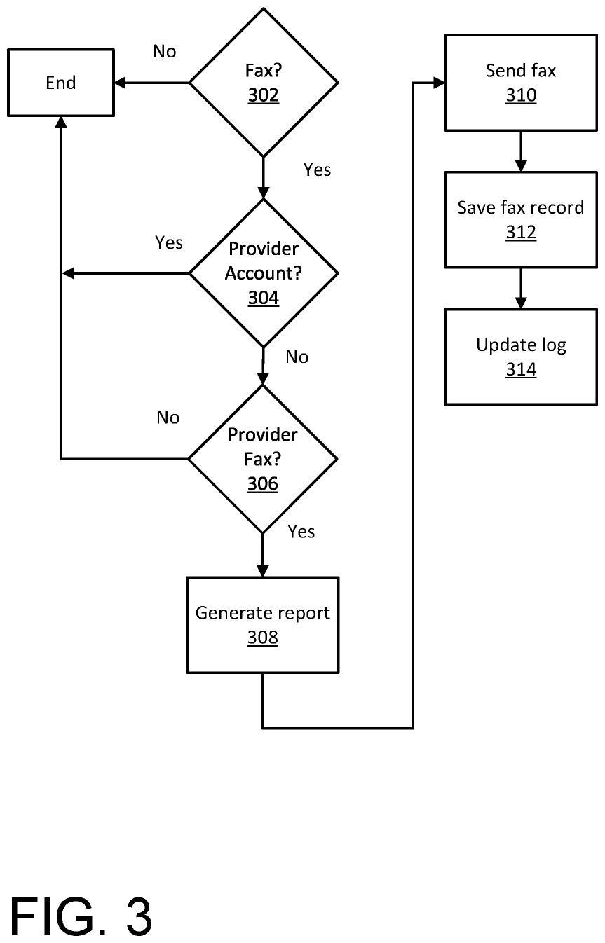

FIG. 3 illustrates a method for sending a status report via fax. In this regard, in block 302, the system 100 determines if the receiving provider account has provided a fax number to the system 100 and whether the receiving account has enabled a fax report feature. In block 302, the system 100 determines whether the receiving healthcare provider (receiving provider) has a referral status enabled in the fax report settings in the user account in the system 100. In block 304, the system 100 determines whether the receiving provider has a user account in the system 100. If no, the system determines whether the referring provider has a fax number in their profile. If yes, in block 308, a summary document such as, for example, a PDF document that includes the referral and the current status of the referral. The status of a referral may include, for example, a workflow step that the referral is currently in. For example, drafts, new, in-review, accepted, declined, canceled, scheduled, attended, no show, complete. Some steps may include a sub step that falls under the step such as, for example a sub step of pending insurance authorization may be under the in-review step. In block 310 the report may be queued to send the report via fax. If the fax fails to send and be properly received by the receiving provider, the system 100 may send the fax multiple times to attempt a satisfactory communication. A In block 312, the fax record including the summary document may be saved in a database or memory. The fax record summary document is a database record that contains data for a fax including the patient name, the date, and a receiving fax number among others. The summary document may be, for example, a PDF file that lists the summary of the referral. The document may include a referring provider name and fax number, receiving provider name and fax number, patient name, gender, birthdate, a reason for a referral, diagnosis and comments. The stored fax record may be associated with a status of the fax transmission. In block 314, a message or indicator is saved in a fax report sent audit record log.

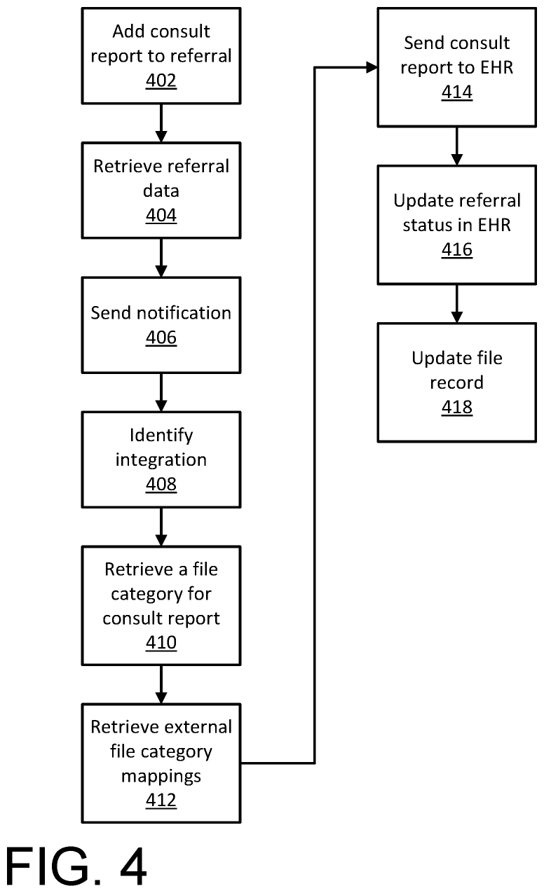

FIG. 4 illustrates a method for sending a consultation report. In block 402, a consultation report is added to a referral by a user manually uploading the report into the system 100. A consultation report may also be added to the referral via polling external EHR application programing interfaces (API), HL7 or another method to determine whether a consultation report is available in the system 100, or if the file is pushed directly by the EHR.

In block 404 the system 100 retrieves the referral details including the provider details. In block 406 the system 100 sends a notification electronically is the referring provider has an account in the system 100. The notification may be sent via fax if the user does not have an account in the system 100.

In block 408, the system determines if system integrations are enabled in the user account. In block 410, the system retrieves a file category to use for the consultation report. A file category is a label that categorizes patient files into groups such as, for example, medical history, orders and prescriptions, test results. These may be defined per customer account. In block 412, the system 100 retrieves external file category mappings used by the integration. In block 414, the system sends a consultation report file to external EHRs via system integration if the system integration is enabled in the account. The system 100 updates referral status in external EHRs via system integration if the feature is enabled in the user account in block 416. In block 418, the system 100 updates the file record in the system with any external identifications returned by the integration for keeping records.

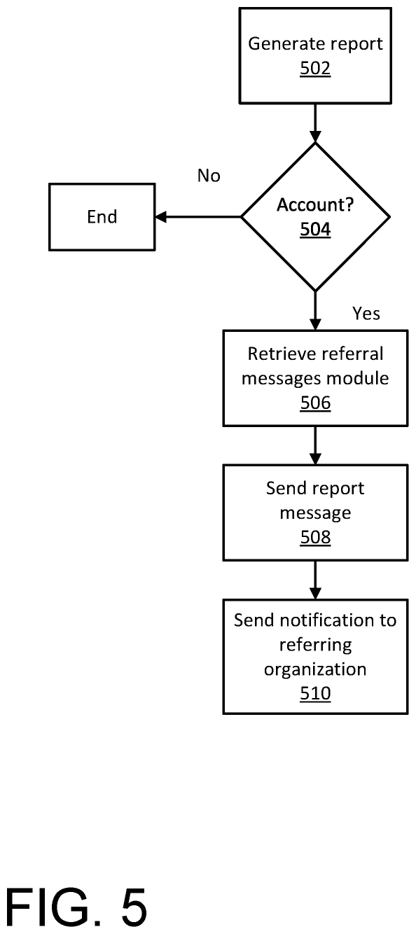

FIG. 5 illustrates a method for sending consultation reports internally in the system 100 via an internal messaging system that may provide communications to a user that has access or an account in the system 100. In block 502 the system 100 generates a report in block 502. The system 100 determines in block 504 whether the user has an account or access to the system 100 in block 504. In block 506, the system 100 retrieves the referral message module. The referral message module generates referral status updates based on the referral information stored in the database module. The referral message module used notification rules and user preferences to determine the timing and frequency of updates based on referral status. In block 508 a consultation report or message is sent in the message module with the consultation report file attached to the message. In block 510, the system 100 sends a notification of the new message to active users in the referring department.

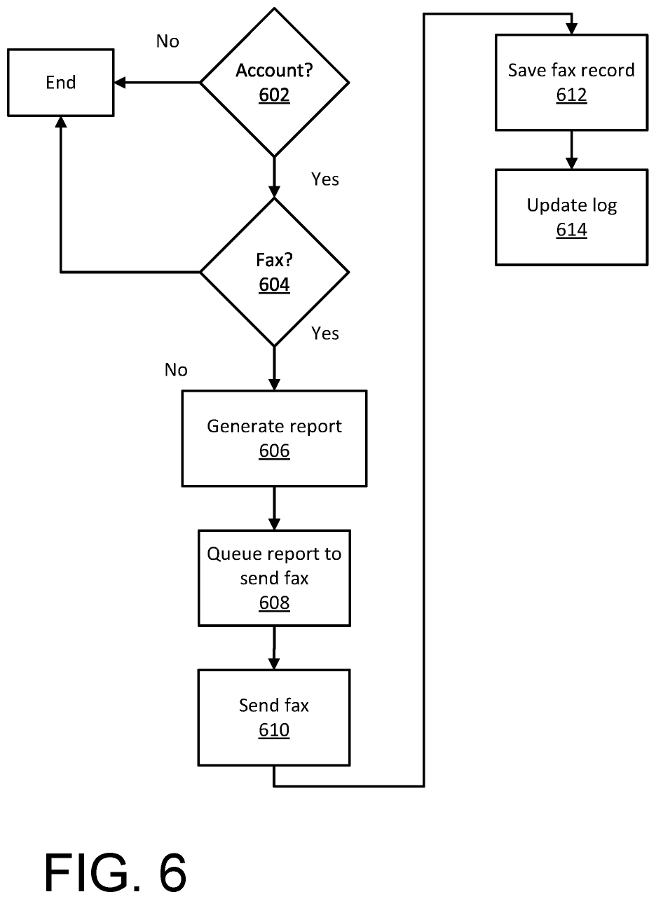

FIG. 6 illustrates a method for sending a consultation report via fax. In block 602, the system 100 determines whether the user has an account or access to the system 100. In block 604 the system 100 determines whether there is a fax number associated with the account. If yes, in block 606, the system 100 generates a consultation report in a format such as, for example a PDF format with a cover page and a consultation report file attached. In block 608 the consultation report is sent to a queue. In block 610, the system 100 sends the fax to the fax number associated with the account. The system 100 may attempt to send the fax a number of times until the recipient receives the fax message in block 612. A copy of the sent PDF is saved in memory and a fax record is saves with the status of the transmission. Examples of a status of transmission include queued for sending via the fax network, pending, when the fax is in the network but has not been received, success in sending the fax, failure in sending the fax. In block 614, the system logs that the fax has been sent into an audit log record.

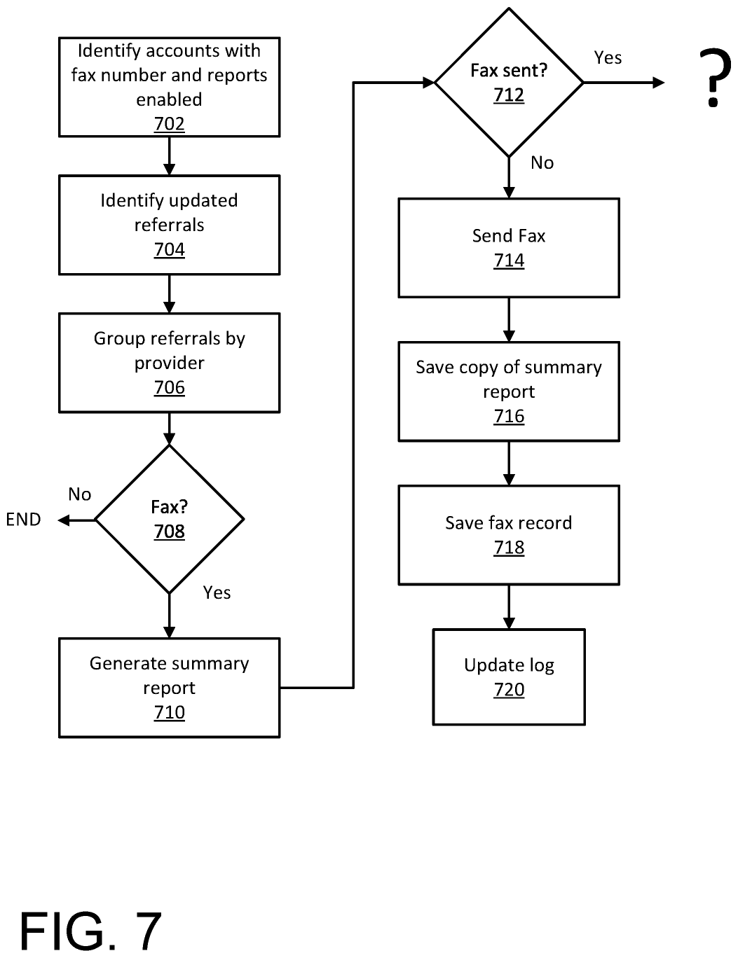

FIG. 7 illustrates an exemplary method for sending automated periodic reports via fax. In block 702, the system 100 identifies user accounts with associated fax numbers and a periodic reports option enabled, In block 704 the system 100 is operative to identify referrals that have been updated within a periodic time period. In block 706 referrals are grouped by associated referring providers. In block 708, the system 100 determines whether the user account has an associated fax number. A summary document such as, for example, a PDF file is generated that includes referrals and the status of referrals that are associated with the referring provider in block 710. The system determines whether a summary has been sent in block 712 to minimize sending multiple reports. In block 714, the report is queued and sent via fax. If the fax is not received, the system 100 may attempt multiple times to send the fax. In block 716, a copy of the report is stored as, for example, a PDF file. In block 718, the system 100 stores a fax record that includes a status of the transmission. In block 720, an audit log record stores an indication that the fax has been successfully transmitted. In block 704 the system 100 is operative to identify updated referrals.

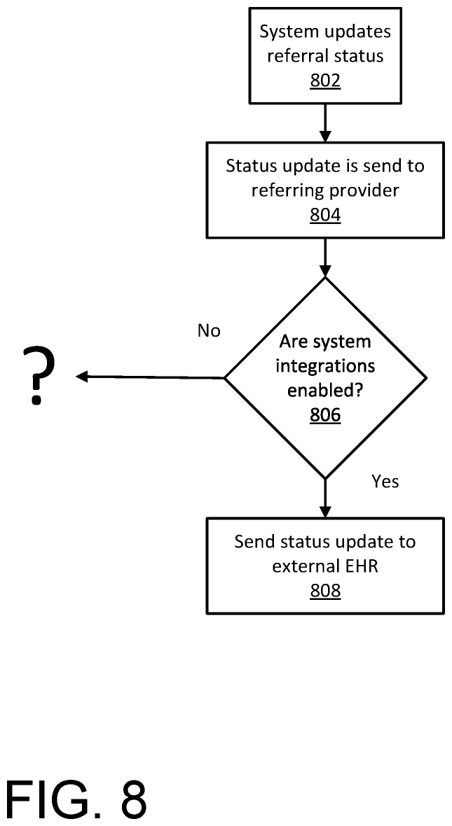

FIG. 8 illustrates a method of sending an update status to an EHR. In block 802, the system 100 updates the referral status or appointment date. In block 804, the status update is sent to the referring provider via fax or by providing the information to a user when a user with a system account assesses the system 100. In block 806, the system determines whether system integrations are enabled for the account. In block 808, the system sends a status update to an external EHR.

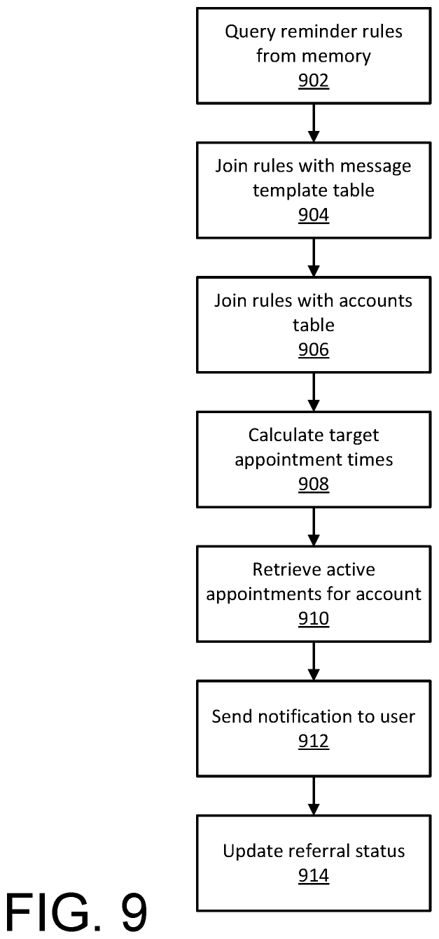

FIGS. 9-14 provide exemplary methods for sending notifications to patients.

Communication rules defined in the system determine when and how a patient communication may be sent to a patient. The communication rules include appointment instructions that notify the patient about an upcoming appointment including details such as, for example, the date, time, location, and healthcare provider. Appointment reminders remind the patient about an upcoming appointment. Appointment follow up rules provide feedback regarding a previous appointment such as, for example, satisfaction surveys or a prompt to reschedule an appointment if an appointment is missed by the patient.

A database stores patient information, including name, contact information, appointment information, appointment schedules, and communication preferences. The database is integrated with EHRs, which confirms that appointment schedules are up-to-date and accurate. The database also allows healthcare providers to manage patient information, update appointment schedules and customize message templates.

The database or memory of the system 100 includes communication rules per account, which include, for example, referrals appointments and patient records. The patient records include, for example, address and corresponding time zones, phone numbers and email addresses.

At a higher lever of operation, the system 100 receives a referral for a patient, the system 100 stores the patient record, including the address, phone numbers, and email of the patient. The system 100 sets the time zone according to the address on record. The system 100 schedules an appointment for the referral.

The system 100 may generate appointment reminders based on the appointment schedules stored in the database or memory. The system 100 uses communication rules to determine a best timing and frequency of reminders based on patient preferences. The system 100 allows healthcare providers to customize reminder messages including language and message content.

The system 100 may process communications by sending appointment reminders to patients via various communication channels, such as SMS or email. The system 100 uses patient preferences to select an effective communication channel for each patient. The system 100 tracks and records patient responses, which allows healthcare providers to monitor patient engagement and adjust the system accordingly.

Patient communications may be sent via a number of methods including SMS or email. A rule has an associated message template that defines the communication contents. This is optimized for the medium. The templates have dynamic variables that are automatically (without human interaction) populated with the data from the referral event, such as appointment dates, location addresses, and attending provider among others. For emails, the messages may contain HTML links with actions such as canceling appointments or confirming appointments.

Rules include triggers that define when a communication should be generated. Examples of rules include, when a referral is added to the system 100, when an appointment is scheduled, when an appointment is confirmed by the patient or canceled, when an appointment is attended or marked as the patient not attending the appointment, a period of time before or after the appointment date, such as, for example, one day before an appointment date, or five minutes after an appointment is confirmed by a patient, and when a referral is marked as completed.

A database or memory of the system 100 includes communication rules per account including referrals, appointments, patient records (addresses and corresponding time zones, phone numbers, and email addresses. Appointment instructions notify the patient of an upcoming appointment, which includes details including a date, time, and location of a provider. Appointment reminders remind the patient about an upcoming appointment. Appointment follow ups provide feedback regarding a past appointment such as, for example, satisfaction surveys when appointments are attended or prompting a reschedule message is an appointment is missed by a patient.

In this regard, FIG. 9 illustrates an exemplary embodiment of a method for sending reminders and follow up messages to a patient. In this regard, reminders and follow up messages may be processed on a periodic schedule such as, for example, a five minute cycle.

In block 902, query reminder rules from the database. Reminder rules include appointment reminders that are based on the appointment schedules stored in the database or memory. The system 100 uses communication rules to determine the best timing and frequency of reminders based on patient preferences. Healthcare provider may customize reminder messages including the language and the content.

In block 904, the system 100 joins the rules with a message template table for each rule. Rules are joined with the accounts table. In block 906 the rules are joined with the accounts table. Each rule contains a database reference to the account that created it and to the message template that rule should use to send a message. In blocks 908-914, the reminder is processed for an account. In this regard, referring to block 908 the system 100 calculates target appointment dates and times to trigger reminders based on the current time and date and the defined frequency of the rule.

In block 910 the system 100 retrieves active scheduled appointments that substantially match the target appointment date and time. In this regard, the system 100 excludes appointments that a previous iteration of the process or another rule has already sent a reminder in, for example, the current day.

In block 912, a reminder notification is sent for each appointment. In block 914, the system 100 updates the referral status with, for example, pending patient confirmation reminders.

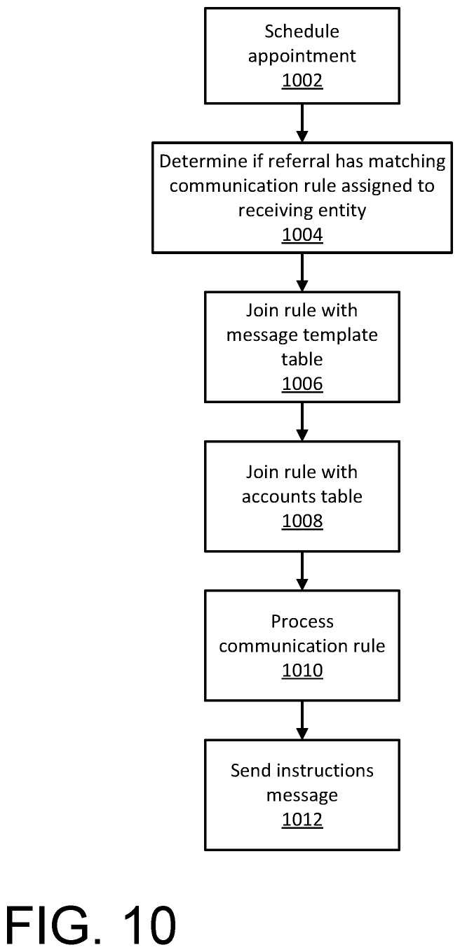

FIG. 10 illustrates an exemplary embodiment of a method for sending appointment instructions. In block 1002, an appointment is scheduled in a referral in the system 100. In block 1004, the system determines if the referring or receiving account in the referral has a matching communication rule defined assigned to the receiving location and department.

In block 1006, the rule is joined with the message template table. In block 1008, the rule is joined with the accounts table. In block 1010, the system processes the communication rule. The system processes the communication rule by parsing each rule. Based on the defined triggers the system finds matching referrals that should send a communication based on the rule. The system joins the message template table to retrieve content that should be send an associated account details. For each referral that matches the rule, the message template is populated with the referral and patient data. Then the message may be sent to the patient via email or SMS as defined in the transport method of the rule. In block 1012, the system sends instructions communication for the appointment.

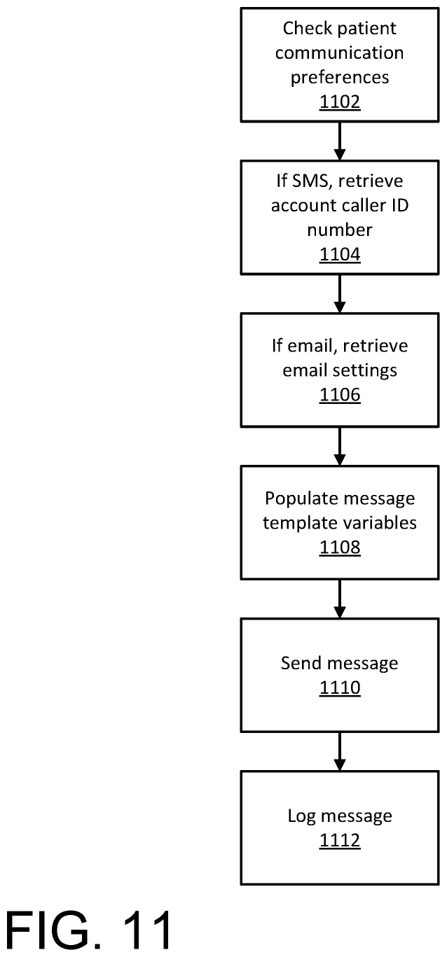

FIG. 11 includes a method for sending a patient communication. In block 1102, the system checks for patient communication preferences, such as, for example, SMS, email, or no communication. In block 1104, the system, when sending an SMS retrieves the account SMS and caller identification number. In block 1106, when sending via email, the system 100 retrieves the email settings such as, for example a reply-to-address or a custom account name. The system 100 populates the message template dynamic variables with corresponding data from the referral and appointment in block 1108. The appointment date, the location address, and the receiving provider name are examples of variables that may be included. The variables may be replaced from the referral data with the actual date, address and name when the communication is sent. In block 1110, the system 100 sends the prepared communication via the selected method, for example, SMS or email. In block 1112, the system 100 logs any communication error notifications for account administrators. If the communication has been sent successfully, the system 100 logs the activity with sent message details.

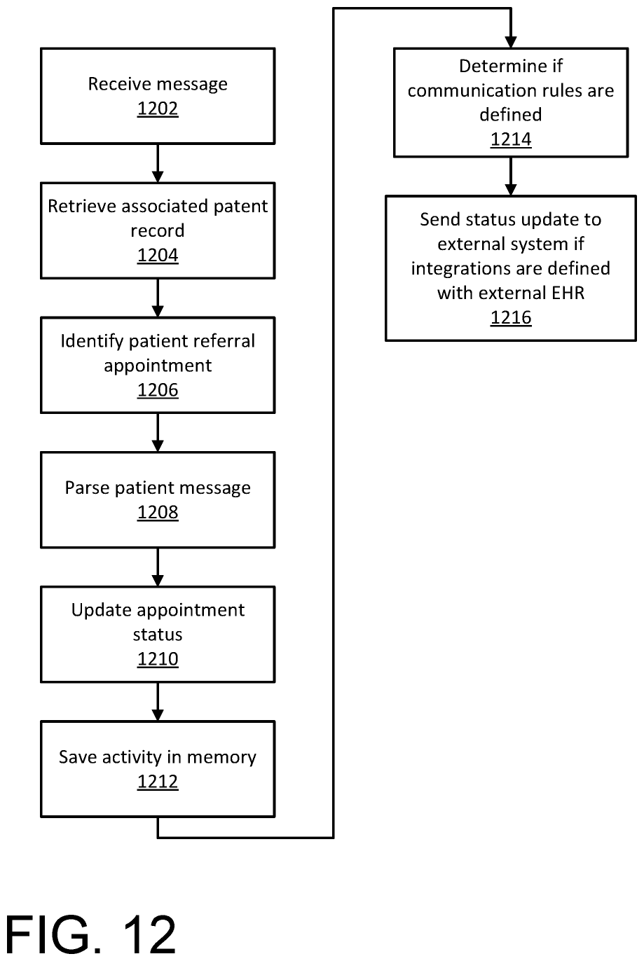

FIG. 12 illustrates an exemplary embodiment of a method for receiving patient responses via SMS. In block 1202, a message is received in an account configured for SMS number responses. In block 1204, the system 100 retrieves a patient record in an account associated with the SMS message. The system 100 retrieves the patient referral appointment that triggered the communication in block 1206. In block 1208 the system 100 parses the patient response text. In this regard, for example, if the message includes the word ‘yes’ the appointment may be marked as confirmed. If the message includes the work ‘no’ the appointment may be marked as cancelled. If the message includes ‘stop’ the patient does not desire to receive SMS communications.

In block 1210, the system 100 updates the appointment status if the received SMS message is confirmed or canceled. In block 1212, the system 100 stores activity in the memory or database, to track the patient response details and the appointment status. If other communication rules are defined with a confirmed or canceled trigger, the system 100 triggers those triggers in block 1214. If the system integrations are defined with an external EHR for the account, the system 100, sends a status update to the external system HER in block 1216.

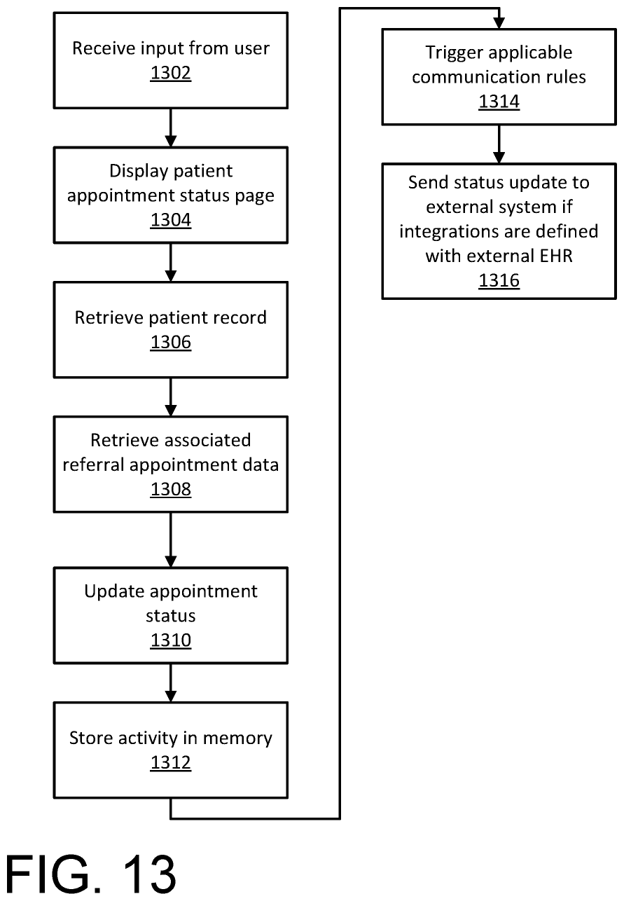

FIG. 13 illustrates an exemplary method for receiving patient responses via email. In block 1302, the patient receives a notification email that includes, for example, HTTP links that allow the user to select one or more links depending on the user preference. The system 100 receives an indication of the link selected by the patient. In block 1304, a browser window is presented to the user that includes the system 100 patient appointment status page. In block 1306, the system 100 identifies a patient record that corresponds to the patient by, for example, data identifiers encoded in the email link that was selected by the patient. In block 1308, the system 100 retrieves information associated with the referral appointment that triggered the communication. In block 1310, the system 100 processes the appointment status if the appointment is confirmed or canceled. In block 1312, the system 100 stores activity in the memory or database to track the patient response details and the appointment status outcome. In block 1314, the system 100 determines if communication rules are defined with a confirmed or canceled trigger. The system 100 triggers those communication rules. In block 1316, the system 100 sends a status update to an external system (a system that is not a part of the system 100) if system integrations are defined with an external EHR for the account.

The system 100 allows for integration with EHRs, which provides accurate and up-to-date appointment schedules. The system 100 provides multiple communication channels for interaction with patients and healthcare providers. The messages may be personalized, which improves patient satisfaction and loyalty. The system is efficient and automated, which reduces the workload of healthcare providers and staff. The system 100 provides real time monitoring and reporting enabling healthcare providers to track patient engagement and adjust the system accordingly.

The referral system described herein provides a system that automatically (without human initiation) notifies patients and providers about the status of referrals. For example, if a patient has a referral appointment, the patient may receive a message notifying the patient of the time and place of the referral appointment. The referral methods of the system generate referral status updates based on the referral information stored in the database. The referral portion of the system uses notification rules and user preferences to determining the timing and frequency of updates based on a referral status.

The computer readable program instructions may be received by a computer or processing device by any suitable process such as, for example, wireless, wired, or optical transmission via a network or other communications devices or processes.

Each block of the illustrations and block diagrams described herein can be implemented by computer readable program instructions, and input to a processor of a computer to carry out or execute the computer readable program instructions.

The descriptions of the various embodiments are disclosed for illustration, but are not intended to limit the disclosed embodiments. Many modifications and variations will be apparent to those of ordinary skill in the art without departing from the scope of the described embodiments.

The system 100 is operative to generate track and send notifications regarding events for referrals from one referring healthcare provider to another receiving healthcare provider and associated appointments. A referral may include, for example a number of data fields with data that reflect the referral. For example, a referral may include fields such as, a receiving provider organization such as, a hospital or hospital system, a receiving provider phone number, a receiving provider fax number (a telephone number that identifies a telephone line connected to a fax machine), a receiving provider name, a receiving provider location, and a receiving provider organizational department. A referral may also include, for example, a referring provider phone number or fax number, a patient name, a patient birthdate, a status of the referral, a date of the referral status last update.

What is claimed is:

1. A method for sending a notification to a recipient, the method comprising:
determining a status of a referral event;
responsive to determining that the status of the referral event, determining communication preferences of the recipient;
populating dynamic variables of a message template with corresponding data associated with the referral event to generate a communication;
sending the communication including information associated with the referral event based on the communication preference;
logging a communication error responsive to a communication error; and
logging that the communication is sent successfully responsive to successfully sending the communication.

2. The method of claim 1, further comprising:
identifying a referring organization associated with a referral event;
retrieving data associated with users associated with the organization; and
sending a notification communication via hypertext transfer protocol (HTTP) web sockets to the user responsive to determining that the user has a system account associated with the user.

3. The method of claim 2, wherein the sending the notification via the HTTP web sockets includes refreshing a browser page to display a referral event status to a user.

4. The method of claim 2, wherein the sending the notification via the HTTP web sockets includes refreshing a browser page to display a number of referrals or number of appointments to a user.

5. The method of claim 1, wherein a communication preference includes a short message system (SMS) message, and
retrieving a caller identification number responsive to determining that the communication preference includes a short message system (SMS) communication preference.

6. The method of claim 1, wherein a communication preference includes an email message, and
the method further includes retrieving email settings responsive to determining that the communication preference includes an email preference.

7. The method of claim 1, wherein the referral event includes patient and provider information, patient information including a name of a patient, contact information of a patient, an appointment schedule of the patient, and a communication preference of the patient.

8. The method of claim 1, further comprising:
sending the communication including information associated with the referral event via short message system (SMS) if the communication preference includes an SMS communication preference;
sending the communication including information associated with the referral event via email if the communication preference includes an email communication preference;
sending the communication including information associated with the referral event via facsimile if the communication preference includes a facsimile communication preference;
sending the communication including information associated with the referral event via electronically via an electronic health records (EHR) system if the communication preference includes an EHR communication preference; and
sending the communication including information associated with the referral via electronically via a system account if the communication preference includes an internal account communication preference.

9. The method of claim 1, wherein a referral event includes an appointment with a healthcare provider.

10. A method for sending a referral event status update, the method comprising:
determining whether a user has a system account associated with the user;
determining whether a facsimile (fax) number associated with the user is saved in the system;
determining whether fax reporting is enabled in the user preferences;
determining whether referral event status reports are enabled in the user preferences;
responsive to determining that fax reporting and referral status reports are enabled in the user preferences, determining whether a fax number associated with a referring provider is saved in the system;
generating a summary referral report;
retrieving the consultation report from system account or electronic health records (EHR) system;
entering the summary referral report into a fax queue; and
sending the summary referral report via fax to the user and the referring provider.

11. The method of claim 10, wherein the summary report includes a report in a PDF format.

12. The method of claim 10, wherein the referral event includes patient and provider information, patient information including a name of the patient, contact information, an appointment schedule, and a communication preference.

13. The method of claim 10, wherein responsive to determining that the user has a system account, the system is operative to:
identifying a referring organization associated with a referral event;
retrieve data associated with users associated with the organization;
identifying an email user preference; and
sending the notification communication via email responsive to identifying that a user prefers receiving an email.

14. The method of claim 13, wherein the referring organization includes a department in a healthcare organization.

15. A method for sending a report by a system, the method comprising:
adding the report to a referral by polling an electronic health records (EHR) system to determine whether the report is available;
retrieving referral information, wherein the referral information includes information associated with a healthcare provider;
determining communication preferences of the recipient; and
sending the communication including information associated with the referral with respect to the communication preference.

16. The method of claim 15, wherein the report includes a consultation report associated with a patient consultation.

17. The method of claim 15, wherein the referral is pushed to the receiving provider electronic health records (EHR) system and the referral status and consultation report are pushed to the referring provider EHR.

18. The method of claim 15, further comprising determining whether system integrations are enabled in an account associated with the user.

19. The method of claim 18, wherein responsive to determining that system integrations are enabled in the account associated with the user:
identifying an integration to use;
retrieving a file category to use for the consultation report;
retrieving file category mappings for the integration;
sending the consultation report file to an EHR via a system integration; and
updating a referral status in the EHR via system integration.

20. The method of claim 19, further comprising updating a file record in the system with identifiers returned by the system integration.
